(19) United States
(12) Patent Application Publication
(10) Pub. No.: US 2026/0275344 A1
(43) Pub. Date: Sep. 17, 2026

(54) CIRCULAR SINGLE-STRANDED DNA MOLECULES FOR RECOMBINASE-BASED GENE EDITING

(71) Applicant: FC IP Holdings, LLC, Dover, DE (US)

(72) Inventors: Keqiang XIE, Cambridge, MA (US); Richard SHAN, Cambridge, MA (US); Hao WU, Lexington, MA (US)

(73) Assignee: FC IP Holdings, LLC, Dover, DE (US)

(21) Appl. No.: 19/472,952

(22) PCT Filed: Apr. 5, 2024
(86) PCT No.: PCT/US2024/023211

§ 371 Date: Oct. 6, 2025

Related U.S. Application Data
(60) Provisional application No. 63/457,420, filed on Apr. 6, 2023.

(51) Int. Cl. C12N 15/113; C12N 1/20; C12N 5/04; C12N 5/071; C12N 9/12; C12N 15/10; C12N 15/90; C12R 1/01

(52) U.S. Cl. CPC C12N 15/113; C12N 1/20; C12N 5/04; C12N 5/0602; C12N 9/1241; C12N 15/1003; C12N 15/102; C12N 15/907; C12N 2310/11; C12N 2310/532; C12N 2800/30; C12N 2830/34; C12N 2830/36; C12R 2001/01

(57) ABSTRACT

Described herein is a circular single-stranded DNA (cssDNA) molecule suitable for recombinase-based gene editing. Also described herein is a double-stranded DNA (dsDNA) molecule and a kit including the same for preparing the cssDNA molecule, as well as a kit and a method for performing genome editing using the cssDNA or the dsDNA.

### CROSS-REFERENCE TO RELATED APPLICATION

The present application claims priority under 35 U.S.C. § 119(e) to U.S. Provisional Patent Application No. 63/457,420, filed Apr. 6, 2023, which is incorporated herein by reference in its entirety.

### SEQUENCE LISTING

The present application contains a Sequence Listing, which has been submitted in XML (ST.26) format and is incorporated herein by reference in its entirety. Said XML copy, created on Apr. 4, 2024, is named “385722-1003WO1_Seq_Listing.xml” and is 13,523 bytes in size.

### BACKGROUND

Recombinase-based gene editing, such as integrase-based gene editing, requires delivering into the host cells high concentrations of double-stranded DNA donor template to achieve efficient integration. The presence of double-stranded DNA in the host cell, however; causes cytotoxicity, which reduces integration efficiency. The dilemma of the need for high donor template concentration and the cytotoxicity caused by the same severely limits the editing efficiency achievable by the current versions of this technology.

There is a need for novel strategies for recombinase-based gene editing, which can achieve high integration efficiency, as well as low cytotoxicity. The present invention addresses this need.

### SUMMARY

In some aspects, the present invention is directed to the following non-limiting embodiments:

### Circular Single-Stranded DNA

In some aspects, the present invention is directed to a circular single-stranded DNA (cssDNA) molecule.

In some embodiments, the cssDNA molecule comprises a recombinase recognition sequence recognized by a recombinase.

In some embodiments, the recombinase is a site-specific recombinase (SSR).

In some embodiments, the site-specific recombinase is a tyrosine integrase, a serine integrase, or a site-specific transposase.

In some embodiments, the recombinase recognition sequence is a locus of X-over P1 (loxP) sequence, a flippase recognition target (FRT) sequence, a rox sequence, an attachment-phage (attP) sequence, an attachment-bacteria (attB) sequence, an attachment-left (attL) sequence, an attachment-right (attR) sequence, a VloxP sequence, a vox sequence, or a transposase recognition inverted terminal repeat (ITR).

In some embodiments, the cssDNA molecule further comprises a payload sequence.

In some embodiments, the payload sequence encodes an RNA molecule or a polypeptide.

In some embodiments, the payload sequence comprises a length of about 1 nucleotide to about 50,000 nucleotides.

In some embodiments, the cssDNA molecule is purified or enriched.

### Double Stranded DNA

In some aspects, the present invention is directed to a double stranded DNA (dsDNA) molecule

In some embodiments, the dsDNA comprises: a template sequence encoding a cssDNA comprising a recombinase recognition sequence recognized by a recombinase; and a cssDNA conversion sequence.

In some embodiments, the double stranded DNA molecule is a linear dsDNA molecule or a circular dsDNA molecule.

In some embodiments, the cssDNA conversion sequence is a sequence originated or derived from an M13 phage f1 origin, an M13 phage replication initiator, an M13 phage replication terminator, or an M13 phage packing signal (PS).

In some embodiments, the dsDNA further comprises a payload sequence, or a payload sequence insertion site for inserting the payload sequence.

In some embodiments, the payload insertion site is a multiple cloning site.

In some embodiments, the ds DNA is one single DNA molecule, or two or more separate DNA molecules.

### Kit

In some aspects, the present invention is directed to a kit.

In some embodiments, the kit comprises: a cssDNA molecule, or a dsDNA molecule and a system that converts the dsDNA molecule to the cssDNA; and a recombinase or a nucleic acid encoding the recombinase.

In some embodiments, the cssDNA molecule or the dsDNA molecule is the same as or similar to those described herein.

In some embodiments, the recombinase recognizes the recombinase recognition sequence of the cssDNA.

In some embodiments, the recombinase is a serine integrase, a tyrosine integrase, or a site-specific transposase.

In some embodiments, the recombinase is Cre, Flp, Dre, SCre, VCre, Vika, B2, B3, KD, ΦC31, Bxb1, RDF, λ, HK022, HP1, γδ, ParA, Gin, R4, TP901-1, TG1, PhiRv1, PhiBT1, SprA, XisF, TnpX, R, A118, spoIVCA, MR11, SCCmec, TndX, XerC, XerD, XisA, Hin, Cin, mrpA, beta, PhiFC1, Fre, Clp, sTre, FimE, HbiF, φFC1, φC1, q 370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), Minos, R1, R2, R3, R4, R5, Mu transposase, Tn3 transposase, Tn5 transposase, Tn7 transposase, Tol2, Sleeping Beauty transposase, or PiggyBac transposase.

In some embodiments, kit comprises the nucleic acid encoding the recombinase, and the nucleic acid encoding the recombinase is an mRNA, a single stranded DNA, or a double stranded DNA.

In some embodiments, the kit further comprises an oligonucleotide fully or partially complementary with the cssDNA molecule.

In some embodiments, the oligonucleotide is a primer that converts the cssDNA molecule to a circular double stranded DNA molecule inside a cell.

In some embodiments, the oligonucleotide comprises a length of about 5 nucleotides to about 200 nucleotides.

In some embodiments, the kit comprises the nucleic acid encoding the recombinase, and the nucleic acid encoding the recombinase is part of the cssDNA molecule or the dsDNA molecule.

In some embodiments, the kit further comprises a component for introducing the cssDNA, and the recombinase or the nucleic acid encoding the recombinase, into the cell.

### System

In some aspects, the present invention is directed to a system.

In some embodiments, the system comprises the kit herein; and a cell to be engineered.

In some embodiments, a genomic DNA or a mitochondrial DNA of the cell comprises a landing pad sequence for the integration of the cssDNA molecule into the genomic DNA or the mitochondrial DNA.

In some embodiments, the landing pad sequence is recognized by the recombinase such that the recombinase opens up the cssDNA molecule and inserts the linearized cssDNA molecule into the genomic DNA or the mitochondrial DNA using the landing pad sequence as an anchor site.

In some embodiments, the landing pad sequence is a locus of X-over P1 (loxP) sequence, a flippase recognition target (FRT) sequence, a rox sequence, an attachment-phage (attP) sequence, an attachment-bacteria (attB) sequence, an attachment-left (attL) sequence, an attachment-right (attR) sequence, a VloxP sequence, a vox sequence, or a transposase recognition inverted terminal repeat (ITR).

In some embodiments, the cell is a bacterial cell, a plant cell, or a mammalian cell.

In some embodiments, the cell is an isolated cell, a cell from a cell line, a primary cell, or a cell in a subject

In some embodiments, the cell is a pluripotent stem cell, an induced pluripotent stem cell (iPSCs), a hematopoietic stem cell (HSCs), a B-cell, a T-cell, a natural killer (NK) cell, or a terminally differentiated non-dividing cell, such as a neuron, a muscle, or a hepatocyte.

### Method for Performing Genetic Engineering

In some aspects, the present invention is directed to a method for performing genetic engineering in a cell.

In some embodiments, the method comprises introducing into the cell: the cssDNA herein; and a recombinase or a nucleic acid encoding the recombinase.

In some embodiments, the recombinase recognizes the recombinase recognition sequence of the cssDNA to integrate the cssDNA into a genomic DNA or a mitochondrial DNA of the cell.

In some embodiments, the recombinase is a serine recombinase, a tyrosine recombinase, or a site-specific transposase.

In some embodiments, the recombinase is Cre, Flp, Dre, SCre, VCre, Vika, B2, B3, KD, ΦC31, Bxb1, RDF, λ, HK022, HP1, γδ, ParA, Gin, R4, TP901-1, TG1, PhiRv1, PhiBT1, SprA, XisF, TnpX, R, A118, spoIVCA, MR11, SCCmec, TndX, XerC, XerD, XisA, Hin, Cin, mrpA, beta, PhiFC1, Fre, Clp, sTre, FimE, HbiF, φFC1, φC1, q 370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), Minos, R1, R2, R3, R4, R5, Mu transposase, Tn3 transposase, Tn5 transposase, Tn7 transposase, Tol2, Sleeping Beauty transposase, or PiggyBac transposase.

In some embodiments, the method further comprises introducing into the cell an oligonucleotide fully or partially complementary to at least a portion of the cssDNA molecule.

In some embodiments, a cellular machinery of the cell converts the cssDNA molecule into a circular double stranded DNA molecule using the oligonucleotide as a primer.

In some embodiments, the oligonucleotide comprises a length of about 5 nucleotides to about 200 nucleotides.

In some embodiments, the method comprises introducing into cells a sense stranded cssDNA, an antisense stranded cssDNA, or a mixture of both.

In some embodiments, the genomic DNA or the mitochondrial DNA of the cell comprises a landing pad sequence for the integration of the cssDNA molecule into the genomic DNA or the mitochondrial DNA.

In some embodiments, the method further comprises culturing the cell under a condition sufficient for the landing pad sequence to be recognized by the recombinase and for the recombinase to open up the cssDNA molecule and insert the linearized cssDNA molecule into the genomic DNA or the mitochondrial DNA using the landing pad sequence as an anchor site.

In some embodiments, the landing pad sequence is a locus of X-over P1 (loxP) sequence, a flippase recognition target (FRT) sequence, a rox sequence, an attachment-phage (attP) sequence, an attachment-bacteria (attB) sequence, an attachment-left (attL) sequence, an attachment-right (attR) sequence, a VloxP sequence, a vox sequence or a transposase recognition inverted terminal repeat (ITR).

In some embodiments, the cell is a bacterial cell, a plant cell, or a mammalian cell.

In some embodiments, the cell is an isolated cell, a cell from a cell line, a primary cell, or a cell in a subject.

In some embodiments, the cell is a pluripotent stem cell, an induced pluripotent stem cell (iPSCs), a hematopoietic stem cell (HSCs), a B-cell, a T-cell, a natural killer (NK) cell, or a terminally differentiated non-dividing cell, such as neuron, muscle, or hepatocyte.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1B: Recombination of attP and attB sites in cssDNA by Bxb1 integrase, in accordance with some embodiments. FIG. 1A: Schematic diagram of the integrase mediated recombination of double-stranded circular DNA or single-stranded circular DNA containing the recognition attP and attB sites. Partially duplexed DNA were prepared by annealing cssDNA with two complementary annealing oligos of attP and attB sites. Bxb1 integrase is expected to catalyze integrative recombination between attP and attB site to produce attL and attR, resulting in two minicircles. The forward and reverse primers were designed in outward direction. Once the attP and attB recombination happens, the forward and reverse primers will amplify a 250 bp band from the resulting minicircle containing attL site. FIG. 1B: The circular DNAs (dsDNA, cssDNA or partially duplexed cssDNA) were delivered to K562 cells with or without Bxb1 mRNA. 24 hours after electroporation, total DNAs were isolated and the existence of minicircle was determined by PCR methods. Top, minicircles were detected when double-stranded phagemid DNA substrate. Bottom, trace amount of minicircles were detected when cssDNA was used as integrase substrate, while significant amount of minicircles were detected when hybrid DNA (cssDNA with duplex at attP and attB site) was used as Bxb1 integrase,

FIGS. 2A-2B: Genome engineering of iPSC cells with integrase and partially duplexed cssDNAs, in accordance with some embodiments. FIG. 2A: Schematic diagram of the study design to examine the integrase mediated genome editing at Rab11a locus in human cells. Cas9 nuclease was guided to the Rab11a target site by the gRNA and induced double strand break. The insertion of “landing pad” at the target site was achieved using oligo containing attB sequence flanked by 50 nt of homology arms at each side of DNA cleavage site as HDR repair template. AttP site followed by EF1a promoter driven GFP DNA cargos were co-delivered with Bxb1 mRNA to examine the efficiency of integrase mediated GFP DNA cargo integration. Double-stranded phagemid containing attP site were used as control. cssDNA was either used directly or to form partially duplexed cssDNA by annealing with a complementary attB oligo. FIG. 2B: Cultured iPSCs were electroporated with Rab11a-RNP, Rab11a-attB oligo, Bxb1 mRNA together with cargo DNA substrate in different forms (dsDNA, cssDNA or partially duplexed DNA). 15 days post electroporation, iPSCs with stable GFP expression were determined by flow cytometry.

FIG. 3 demonstrates that the recombinase-based gene editing strategy herein also works when transposase is used as the recombinase, in accordance with some embodiments. K562 cells were mock transfected, or transfected with a cssDNA including EF1a-GFP encoding sequence flanked by transposase recognition inverted terminal repeat (ITR) alone, or with the cssDNA and an mRNA encoding the codon-optimized NLS tagged PiggyBac. At 21 days post transfection, expressions of GFP in the cells were detected. At day 21 post electroporation, ~39.30% of GFP reporter expression was observed from stably integrated EF1a-GFP transgene, with only ~2.37% of GFP expression from the cssDNA donor template alone group, indicating substantial stable genomic integration of the reporter transgene by NLS tagged PiggyBac Transposase.

FIG. 4 demonstrates that recombinase-based gene editing strategy, when using transposase as the recombinase, can edit primary T cells, in accordance with some embodiments. Primary T cells were transfected with a cssDNA including EF1a-GFP encoding sequence flanked by transposase recognition inverted terminal repeat (ITR) alone, or with the cssDNA and an mRNA encoding the codon-optimized NLS tagged PiggyBac. At day 14 post electroporation, ~7.54% of GFP reporter expression was observed from stably integrated EF1a-GFP transgene, with only ~0.16% of GFP expression from the cssDNA donor template alone group, indicating substantial stable genomic integration of the reporter transgene by NLS tagged PiggyBac Transposase.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

The present study made the unexpected discovery that, while the recombinases used in genetic editing are not known to recognize single stranded DNA (ssDNA) molecules or integrate ssDNA molecules into double stranded host DNA (e.g., genomic DNA or mitochondrial DNA), the recombinases can integrate circular single-stranded DNA (cssDNA) molecules including recombinase recognition sequences into the host DNA.

Most unexpectedly, the efficiency of this cssDNA integration, while relatively low, can be greatly enhanced with the presence of single-stranded oligonucleotides that duplex with the cssDNA molecules. For example, in the presence of a duplexing single-stranded oligonucleotide, a non-limiting exemplary recombinase, Bxb1, was able to achieve a surprisingly high integration efficiency with a cssDNA molecule, substantially exceeding the integration efficiency with a corresponding circular double-stranded DNA molecule.

Without wishing to be bound by theories, it is hypothesized that, after the cssDNA molecules enter the host cell, the cssDNA molecules pass through the cytosol and enters the cell nucleus, where cellular machineries within the nucleus (such as the DNA replication machineries) convert the cssDNA into circular double-stranded DNA molecules. The recombinases then integrate the newly produced double-stranded DNA molecules into the genomic DNA. The presence of the duplexing single-stranded oligonucleotides provides the cellular machineries with primers, which speeds up the conversion from the cssDNA to the corresponding double stranded DNA, thus improving the editing efficiencies. Furthermore, since both the cssDNA molecules for integration and the duplexing single-stranded oligonucleotides pass through the cytosol as single-stranded DNA molecules, they do not alert the host cells as invasive cytosolic DNA or activate innate immunity, which may at least partially explain the significantly higher editing efficiency.

Accordingly, in some aspects, the present invention is directed to a cssDNA molecule, such as a cssDNA molecule suitable for being integrated into a host DNA by a recombinase. cssDNA molecules are relatively difficult to edit when compared to double-stranded DNA molecules. As such, cssDNA molecules are often edited as double-stranded DNA molecules, before being converted into the cssDNA molecules.

Accordingly, in some aspects, the present invention is directed to a double stranded DNA molecule, such as a double stranded DNA molecule configured to produce the cssDNA molecule herein. In some aspects, the present invention is directed to a kit for generating the cssDNA molecule herein, such as from the double-stranded DNA molecule herein.

The cssDNA molecule herein, when properly paired with a recombinase and/or a duplexing oligonucleotide, can be used to edit host DNA, such as the genomic DNA or mitochondrial DNA of a host cell.

Accordingly, in some aspects, the present invention is directed to a kit for performing genome editing.

The cssDNA molecule herein, when properly paired with a recombinase and/or a duplexing oligonucleotide, as well as a suitable host cell, can be used to produce genetically engineered cells.

Accordingly, in some aspects, the present invention is directed to a system for preparing a genetically engineered cell.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

### Circular Single-Stranded DNA Molecule

In some aspects, the present invention is directed to a circular single-stranded DNA (cssDNA) molecule.

In some embodiments, the cssDNA molecule is configured to be integrated into a host DNA molecule, such as a genomic DNA molecule or a mitochondrial DNA molecule of a host cell, by a recombinase.

In some embodiments, the cssDNA molecule includes a recombinase recognition sequence.

In some embodiments, the terms “recombinase” refers to an enzyme that is able to form a complex with a circular double-stranded DNA molecule and a DNA molecule, such as a genomic DNA or a mitochondrial DNA, of a host cell, and integrates the circular double-stranded DNA into the host DNA. In some embodiments, the recombinase specifically recognizes the recombinase recognition site, and “grabs” the circular double-stranded DNA molecule by the recombinase recognition site and bring the double-stranded DNA molecule to the host DNA.

In some embodiments, the recombinase herein does not actually recognize the single-stranded recombinase recognition sequence directly. Rather, the recombinase only recognizes the recombinase recognition sequence when it is converted into a double-stranded DNA sequence. In some embodiments, the recombinase recognition sequence in the cssDNA molecule is either strand of the corresponding double-stranded DNA recombinase recognition sequence recognizable by the recombinase.

In some embodiments, the recombinase is a non-specific recombinase. In some embodiments, the term “non-specific recombinase” refers to a recombinase that they do not recognize specific sequences on the host DNA, even though they do recognize the recombinase recognition site on the circular double-stranded DNA molecule specifically. Non-limiting examples of non-specific recombinase include most natural existing transposases which may or may not have preferences in the host DNA sequences for integration, but lacks specificity for any particular DNA sequences.

In some embodiments, the recombinase is a site-specific recombinase (SSR). In some embodiments, the site-specific recombinase specifically recognizes both the recombinase recognition site on the circular double-stranded DNA molecule and specific sequences on the host DNA. As such, the site-specific recombinase is able to integrate the circular double-stranded DNA molecule at specific locations in the host DNA. In some embodiments, site-specific recombinases can be categorized into tyrosine integrases, serine integrases and genetically engineered site-specific transposases.

Non-limiting examples of site-specific recombinases include Cre, Flp, Dre, SCre, VCre, Vika, B2, B3, KD, ΦC31, Bxb1, RDF, λ, HK022, HP1, γδ, ParA, Gin, R4, TP901-1, TG1, PhiRv1, PhiBT1, SprA, XisF, TnpX, R, A118, spoIVCA, MR11, SCCmec, TndX, XerC, XerD, XisA, Hin, Cin, mrpA, beta, PhiFC1, Fre, Clp, sTre, FimE, HbiF, φFC1, φC1, q 370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), Minos, R1, R2, R3, R4, R5, and the like. Non-limiting examples of site-specific recombinases further include modified or engineered transposases that possess site-specificity, such as modified or engineered Mu transposase, Tn3 transposase, Tn5 transposase, Tn7 transposase, Tol2, Sleeping Beauty transposase or PiggyBac transposase and the like.

In some embodiments, the recombinase recognition sequence in the cssDNA molecule is a recognition sequence for a site-specific recombinase (SSR).

In some embodiments, the cssDNA molecule, with the aid of the recombinase, such as the site-specific recombinase, can be integrated into a DNA molecule, such as a genomic DNA molecule or a mitochondrial DNA molecule, of a host cell, such as in a site-specific manner.

In some embodiments, the recombinase, such as the site-specific recombinase, recognizes the recombinase recognition sequence in the cssDNA molecule (or the dsDNA molecule converted from the cssDNA molecule).

In some embodiments, the recombinase, such as the site-specific recombinase, recognize both the recombinase recognition sequence in the cssDNA molecule (or the dsDNA molecule converted from the cssDNA molecule), and a “landing pad” sequence in the DNA molecule in the host cell.

In some embodiments, the recombinase, such as the site-specific recombinase, “grabs” the cssDNA molecule (or the dsDNA molecule converted from the cssDNA molecule) by the recombinase recognition sequence, and brings the cssDNA (or the dsDNA molecule converted from the cssDNA molecule) in close proximity to the host cell DNA molecule using the landing pad sequence as an anchor, and integrate the cssDNA (or the dsDNA molecule converted from the cssDNA molecule) into the host cell DNA in a site-specific manner.

In some embodiments, the recombinase recognition sequence is a locus of X-over P1 (loxP) sequence, a flippase recognition target (FRT) sequence, a rox sequence, an attachment-phage (attP) sequence, an attachment-bacteria (attB) sequence, an attachment-left (attL) sequence, an attachment-right (attR) sequence, a VloxP sequence, a vox sequence, or transposase recognition inverted terminal repeats (ITRs). In some embodiments, the recombinase recognition sequence is a sense strand, an antisense strand, or a combination of both.

In some embodiments, the cssDNA molecule further includes a payload sequence.

In some embodiments, the payload sequence encodes an RNA molecule or a polypeptide.

In some embodiments, the payload sequence in the cssDNA molecule is a sense strand, an antisense strand, or a combination of both.

In some embodiments, a length of the payload sequence ranges from about 1 base to about 50,000 nucleotides, such as from about 10 nucleotides to about 20,000 nucleotides, from about 50 nucleotides to about 10,000 nucleotides, from about 100 nucleotides to about 5,000 nucleotides, or from about 200 nucleotides to about 2,000 nucleotides. In some embodiments, the length of the payload sequence is about 1 base, about 10 nucleotides, about 20 nucleotides, about 50 nucleotides, about 100 nucleotides, about 200 nucleotides, about 500 nucleotides, about 1,000 nucleotides, about 2,000 nucleotides, about 5,000 nucleotides, about 10,000 nucleotides, about 20,000 nucleotides, about 50,000 nucleotides, or any ranges therebetween.

In some embodiments, the cssDNA molecule is purified or enriched. In some embodiments, the cssDNA molecule is considered purified or enriched if the molar ratio between the cssDNA molecule and the total DNA molecules in a system is about 50% or higher, such as about 60% or higher, about 70% or higher, about 80% or higher, about 90% or higher, about 95% or higher, about 98% or higher, or about 99% or higher.

In some embodiments, the cssDNA is chemically or enzymatically synthesized, such as in vitro. In some embodiments, the cssDNA is prepared from one or more double-stranded DNA molecule, such as using the M13 phage system.

Double Stranded DNA Molecule and Kit Including the Same for Preparing cssDNA

In some aspects, the present invention is directed to a double stranded DNA (dsDNA) molecule.

In some embodiments, the dsDNA molecule is a DNA molecule for preparing the cssDNA herein.

In some embodiments, the dsDNA molecule includes a template sequence encoding the cssDNA; and a cssDNA conversion element for converting the dsDNA molecule into the cssDNA molecule.

In some embodiments, the double stranded DNA molecule is a linear dsDNA molecule or a circular dsDNA molecule.

In some embodiments, the cssDNA conversion sequence comprises a replication origin and/or a packing signal originated or derived from the genomic DNA of a cssDNA virus, such as a DNA packaging element from the genomic DNA of a filamentous bacteriophage. While inside infected host cells, the cssDNA viral proteins, such as the filamentous bacteriophage proteins, replicate the circular single-stranded genomic DNA by recognizing the replication origin, and package the circular single-stranded genomic DNA by recognizing the packing signal. Such mechanisms have been utilized to mass produce cssDNA from double-stranded DNA molecules (see e.g., Xie et al., bioRxiv 2022.12.01.518578, Cha et al., Advanced Functional Materials Volume 31, Issue 35, Aug. 26, 2021, and Shepherd et al., Sci Rep 9, 6121 (2019)) Accordingly, in some embodiments, the cssDNA conversion sequence comprises filamentous bacteriophage replication original, or a packing signal.

In some embodiments, the cssDNA conversion sequence comprises an M13 phage f1 origin, an M13 phage replication initiator, an M13 phage replication terminator, or an M13 phage packing signal (PS).

In some embodiments, the dsDNA molecule further includes a payload sequence, or a payload sequence insertion site for inserting the payload sequence. In some embodiments, the payload sequence is the same as or similar to those as described for the cssDNA molecule, except that it is double-stranded here.

In some embodiments, the payload insertion site is a multiple cloning site, such as a multiple cloning site including restriction enzyme cutting sequences similar to those found in commercially available plasmid vectors.

It is worth noting that the dsDNA molecule herein does not have to be one single dsDNA molecule, as the construction of cssDNA molecules sometimes involves the use of multiple double-stranded DNA molecules, such as described in Shepherd et al. (Sci Rep 9, 6121 (2019)). Accordingly, in some embodiments, the dsDNA molecule is one single DNA molecule, or two or more separate DNA molecules.

In some aspects, the present invention is directed to a kit for preparing the cssDNA molecule herein.

In some embodiments, the kit includes the dsDNA molecule herein; and a component for converting the dsDNA molecule into the cssDNA. Non-limiting examples of such components include an M13 helper plasmid (see e.g., Xie et al., bioRxiv 2022.12.01.518578).

### Kit for Performing Genome Editing

In some aspects, the present invention is directed to a kit for performing genome editing in a cell. In some embodiments, genome editing includes editing a genomic DNA, or a mitochondrial DNA of a host cell, such as a mammalian cell, a plant cell or other type of eukaryotic cells, or a bacterial cell.

In some embodiments, the kit includes the cssDNA molecule herein, the dsDNA molecule herein, or the kit for preparing the cssDNA molecule herein; and a recombinase or a nucleic acid encoding the recombinase.

In some embodiments, the recombinase recognizes the recombinase recognition sequence of the cssDNA. In some embodiments, the recombinase recognizes the recombinase recognition sequence of the cssDNA (or the circular double-stranded DNA converted from the cssDNA by the host cell), and inserts the cssDNA (or the circular double-stranded DNA converted from the cssDNA by the host cell) into the host DNA. In some embodiments, the recombinase (such as a site-specific recombinase), recognizes both the recombinase recognition sequence of the cssDNA (or the circular double-stranded DNA converted from the cssDNA by the host cell) and a “landing pad” sequence on the host DNA, and inserts the cssDNA (or the circular double-stranded DNA converted from the cssDNA by the host cell) into the host DNA using the landing pad sequence as an anchor point.

In some embodiments, the recombinase is the same as or similar to those described elsewhere herein. In some embodiments, the recombinase is a serine integrase, a tyrosine integrase, or a transposase (such as a site-specific transposase). In some embodiments, the recombinase is Cre, Flp, Dre, SCre, VCre, Vika, B2, B3, KD, ΦC31, Bxb1, RDF, λ, HK022, HP1, γδ, ParA, Gin, R4, TP901-1, TG1, PhiRv1, PhiBT1, SprA, XisF, TnpX, R, A118, spoIVCA, MR11, SCCmec, TndX, XerC, XerD, XisA, Hin, Cin, mrpA, beta, PhiFC1, Fre, Clp, sTre, FimE, HbiF, φFC1, φC1, q 370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), Minos, R1, R2, R3, R4, R5, or the like. In some embodiments, the recombinase is Mu transposase, Tn3 transposase, Tn5 transposase, Tn7 transposase, Tol2, Sleeping Beauty transposase, PiggyBac transposase, or the like. Natural existing transposases normally lacks site-specificity. Transposases, however; can become site-specific via modifications and/or engineering.

In some embodiments, the kit includes the nucleic acid encoding the recombinase, and the nucleic acid encoding the recombinase is an mRNA, a single stranded DNA, or a double stranded DNA. In some embodiments, the nucleic acid encoding the recombinase is incorporated into the cssDNA molecule herein. In some embodiments, the nucleic acid encoding the recombinase is not incorporated into the cssDNA molecule herein, but is on a separate polynucleotide molecule.

In some embodiments, the kit further includes an oligonucleotide fully or partially complementary with the cssDNA molecule. In some embodiments, the oligonucleotide is a DNA, an RNA, a DNA-RNA hybrid, or chemically modified nucleic acid.

In some embodiments, the oligonucleotide and the cssDNA has a complementarity of about 80% or higher, such as about 85% or higher, about 90% or higher, about 95% or higher, about 98% or higher, or 100% (i.e., the oligonucleotide is fully complementary with the cssDNA). As used herein, an oligonucleotide shorter than the cssDNA is 80% complementary with the cssDNA if 80% of the nucleotides in the oligonucleotide is complementary with the cssDNA, and the oligonucleotide is fully complementary with the cssDNA if all the nucleotides in the oligonucleotide is complementary with the cssDNA.

In some embodiments, a length of the oligonucleotide ranges from about 5 nucleotides to about 200 nucleotides, such as from about 8 nucleotides to about 160 nucleotides, from about 10 nucleotides to about 120 nucleotides, from about 15 nucleotides to about 100 nucleotides, or from about 20 nucleotides to about 80 nucleotides. In some embodiments, the length of the oligonucleotide is about 5 nucleotides, about 8 nucleotides, about 10 nucleotides, about 12 nucleotides, about 15 nucleotides, about 20 nucleotides, about 25 nucleotides, about 30 nucleotides, about 40 nucleotides, about 50 nucleotides, about 60 nucleotides, about 70 nucleotides, about 80 nucleotides, about 90 nucleotides, about 100 nucleotides, about 120 nucleotides, about 140 nucleotides, about 160 nucleotides, about 180 nucleotides, about 200 nucleotides, or any ranges therebetween.

In some embodiments, the oligonucleotide is a primer for converting the cssDNA molecule into a circular double stranded DNA molecule in the cell. In some embodiments, the oligonucleotide serves as a primer for the DNA replication machineries of the host cell, such that the DNA replication machineries can convert the cssDNA molecule into the circular double stranded DNA molecule more easily, such as in the nucleus of the cell.

In some embodiments, the kit comprises the nucleic acid encoding the recombinase, and wherein the nucleic acid encoding the recombinase is part of the cssDNA molecule or the dsDNA molecule.

In some embodiments, the kit further includes a component for introducing the cssDNA, and the recombinase or the nucleic acid encoding the recombinase into the cell. Non-limiting examples of such component includes lipid nanoparticles, metal nanoparticles, micelles, microinjectors, electroporation cuvettes, exosome and the like.

In some embodiments, the recombinase, and the recombinase recognition sequence are matched such that the recombinase recognizes both the recombinase recognition sequence, and is able to integrate the cssDNA molecule herein (or the double-stranded conversion product thereof made by the host cell) into the host DNA.

### System for Preparing Genetically Engineered Cell

In some aspects, the present invention is directed to a system for preparing a genetically engineered cell.

In some embodiments, the system includes the kit for performing genome editing herein; and a cell to be engineered.

In some embodiments, a genomic DNA or a mitochondrial DNA of the cell includes a landing pad sequence for the integration of the cssDNA molecule (or the double-stranded conversion product made by the host cell).

In some embodiments, the landing pad sequence is recognized by the recombinase such that the recombinase opens up the cssDNA molecule and inserts the linearized cssDNA molecule (or the double-stranded conversion product made by the host cell) into the genomic DNA or the mitochondrial DNA using the landing pad sequence as an anchor site.

In some embodiments, the landing pad sequence is a locus of X-over P1 (loxP) sequence, a flippase recognition target (FRT) sequence, a rox sequence, an attachment-phage (attP) sequence, an attachment-bacteria (attB) sequence, an attachment-left (attL) sequence, an attachment-right (attR) sequence, a VloxP sequence, a vox sequence, or a transposase recognition inverted terminal repeat (ITR).

In some embodiments, the “landing pad” sequence (i.e., the targeting genome site for integration) is any location that is recognized by the recombinases herein.

In some embodiments, the “landing pad” sequence is inserted into the host DNA, or can be the locus of a mutated gene.

In some embodiments, the “landing pad” is inserted into the host DNA via the site-specific nuclease selected from the group, such as a Cas nuclease, a zinc-finger nuclease (ZFN), a meganuclease, a transcription activator-like effector nuclease (TALEN), or the like. In some embodiments, the “landing pad” sequence is inserted via guided prime editors technology without double strand break. Non-limiting examples of prime editors technology include those using reverse transcriptase (RT) fused Cas 9 H840A nickase (Cas9n (H840A)), the genome editing is achieved using a prime-editing guide RNA (pegRNA), or the like.

In some embodiments, the cell is a bacterial cell, a plant cell, or a mammalian cell.

In some embodiments, the cell is an isolated cell, a cell from a cell line, a primary cell, or a cell in a subject.

In some embodiments, the cell is a pluripotent stem cell, an induced pluripotent stem cell (iPSCs), a hematopoietic stem cell (HSCs), a B-cell, a T-cell, a natural killer (NK) cell, or a terminally differentiated non-dividing cell, such as neuron, muscle, or hepatocyte.

In some embodiments, the recombinase, the recombinase recognition sequence, and the “landing pad” sequence are matched such that the recombinase recognizes both the recombinase recognition sequence and the “landing pad” sequence, and is able to integrate the cssDNA molecule herein (or the double-stranded conversion product thereof made by the host cell) into the host DNA.

### Method of Performing Genetic Engineering

In some embodiments, the present invention is directed to a method of performing genetic engineering in a cell, such as editing the genomic DNA or the mitochondrial DNA of the cell by inserting the cssDNA molecule (or the double-stranded conversion product thereof made by the cell) into the DNA of the cell.

In some embodiments, the method includes introducing to the cell: the cssDNA molecule herein; and a recombinase or a nucleic acid encoding the recombinase.

In some embodiments, the recombinase recognizes the recombinase recognition sequence of the cssDNA, and opens up and inserts the cssDNA into a genomic DNA or a mitochondrial DNA of the cell.

In some embodiments, the recombinase is a serine recombinase, a tyrosine recombinase, or a site-specific transposase. In some embodiments, recombinase is Cre, Flp, Dre, SCre, VCre, Vika, B2, B3, KD, ΦC31, Bxb1, RDF, λ, HK022, HP1, γδ, ParA, Gin, R4, TP901-1, TG1, PhiRv1, PhiBT1, SprA, XisF, TnpX, R, A118, spoIVCA, MR11, SCCmec, TndX, XerC, XerD, XisA, Hin, Cin, mrpA, beta, PhiFC1, Fre, Clp, sTre, FimE, HbiF, φFC1, φC1, q 370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), Minos, R1, R2, R3, R4, R5, Mu transposase, Tn3 transposase, Tn5 transposase, Tn7 transposase, Tol2, Sleeping Beauty transposase or PiggyBac transposase. In some embodiments, the method further includes introducing into the cell an oligonucleotide fully or partially complementary with the cssDNA molecule, such as the duplexing oligonucleotide described elsewhere herein.

In some embodiments, a cellular machinery of the cell converts the cssDNA molecule into a circular double stranded DNA molecule using the oligonucleotide as a primer.

In some embodiments, the method further includes introducing into cells either sense or antisense stranded cssDNA, or both sense and antisense stranded cssDNA. In some embodiments, only the sense stranded cssDNA is introduced. In some embodiments, only the antisense strand is introduced. In some embodiments, both the sense strand and the antisense strand are introduced.

In some embodiments, the genomic DNA or the mitochondrial DNA of the cell includes a landing pad sequence for the integration of the cssDNA molecule. In some embodiments, the cell, the “landing pad” sequence, and/or the method of introducing the “landing pad” sequence to the cell is the same as or similar to those described elsewhere herein.

In some embodiments, the landing pad sequence is recognized by the recombinase such that the recombinase opens up the cssDNA molecule and inserts the linearized cssDNA molecule into the genomic DNA or the mitochondrial DNA using the landing pad sequence as an anchor site.

In some embodiments, the landing pad sequence is a locus of X-over P1 (loxP) sequence, a flippase recognition target (FRT) sequence, a rox sequence, an attachment-phage (attP) sequence, an attachment-bacteria (attB) sequence, an attachment-left (attL) sequence, an attachment-right (attR) sequence, a VloxP sequence, a vox sequence, or a transposase recognition inverted terminal repeat (ITR).

In some embodiments, the cell is a bacterial cell, a plant cell, or a mammalian cell.

In some embodiments, the cell is an isolated cell, a cell from a cell line, a primary cell, or a cell in a subject.

In some embodiments, the cell is a pluripotent stem cell, an induced pluripotent stem cell (iPSCs), a hematopoietic stem cell (HSCs), a B-cell, a T-cell, a natural killer (NK) cell, or a terminally differentiated non-dividing cell, such as neuron, muscle, or hepatocyte.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Integrase-Mediated Genome Engineering Using Partially Duplexed Circular Single-Stranded DNA as Donor Template

DNA engineering is the centerpiece of synthetic biology. Genome editing tools have been rapidly expanding and quickly adapted in basic research and therapeutic applications. With more comprehensive understanding of the human genome as well as the etiology of various disorders, genetic engineering has emerged as a powerful tool to treat diseases and to conduct biomedical research. Of the numerous methods for genetic manipulation, integrase technology has been well studied but not utilized to its full potential. Integrases are site-specific recombinase (SSR) that are involved in the integration of DNA into host cell genomes. They recognize unique short DNA sequences in both the host genome and the exogenous DNA species and create hybrid sequence by inserting the transgene into the host genome.

Serine integrases, a subfamily of integrases, are encoded by temperate bacteriophages and catalyze their integration into bacterial genomes through recombination of attP (phage) and attB (bacteria) attachment sites, generating attL (left) and attR (right) sites. Importantly, recombination of attP and attB is highly directional, it is reversible only in the presence of a single accessory protein called recombination directionality factor (RDF), making them a valuable tool for genetic engineering, as they induce permanent changes to the genome. Recombination by serine integrases normally only requires the integrase protein and small att sites (~50 bp), making these proteins powerful tools for genome engineering (Merrick et al., ACS Synth Biol, 7(2), 299-310, 2018).

Serine integrases were first reported to engineer Streptomyces genomes at innate attB sites (Boccard et al., Plasmid, 21(1), 59-70, 1989; Kuhstoss et al., Gene, 97(1), 143-146, 1991). These enzymes have been widely utilized to engineer a number of organisms, including Chinese Hamster Ovary (CHO) cells (Andreas et al., Nucleic Acids Res, 30(11), 2299-2306, 2002) and Drosophila embryos (Groth et al., Genetics, 166(4), 1775-1782, 2004). Genome insertion of transgenes using serine integrases offers a number of advantages comparing to other genome engineering methods. In comparison to homologous recombination-based methods including those that employ homing endonucleases such as zinc finger nucleases (ZFN), transcription activator-like effector nucleases (TALEN) and CRISPR-Cas9, integration is mediated by only one enzyme without relying on host factors for DNA insertion (Gaj et al., Trends Biotechnol, 31(7), 397-405, 2013). Unlike transposons and retroviruses, integration can be targeted to a specific locus known to have minimal positional effects on transgene expression (Vrljicak et al., G3 (Bethesda), 6(4), 805-817, 2016). Transgenes integrated by serine integrases though attP×attB recombination is unidirectional and cannot be inverted or remobilized without the presence of a cognate RDF. Genome engineering approaches that use serine integrases can be broadly grouped into those that integrate DNA into pre-existing genomic loci (pseudo sites), those that use a “landing pad” (a single att site integrated into the genome), and recombinase-mediated cassette exchange.

To date four serine integrases derived from phages PhiC31 (Andreas et al., Nucleic Acids Res, 30(11), 2299-2306, 2002), PhiBT1 (Xu et al., Nucleic Acids Res, 36(1), e9, 2008), Bxb1 (Russell et al., Biotechniques, 40(4), 460, 462, 464, 2006; Yamaguchi et al., PLoS One, 6(2), e17267, 2011) and R4 (Olivares et al., Gene, 278(1-2), 167-176, 2001; Yamaguchi et al., PLoS One, 6(2), e17267, 2011) have been shown to be capable of promoting site-specific integration of DNA into mammalian genomes. Among them, PhiC31 and Bxb1 integrases are extensively used in genetic engineering and biotechnology research. Both can insert long DNA sequences which are beyond the capability of other editing tools, but it needs an existing attB site as the “landing pad”. The current strategy of using integrases for gene editing is to first insert an attB site into the genome at a desired location, e.g., AAVS1. This step is usually done by a nuclease such as Cas9 (or its modified version). Next, a plasmid containing the transgene sequence and attP will be introduced into the cell, along with PhiC31 or Bxb1 integrase, which in turn integrates the transgene into the host genome at the attB site. Indeed, CRISPR prime editing technology has been used to place the prerequisite “landing pad” into the mammalian cell genome. When combined with a serine integrase (i.e., Bxb1 integrase), this technology enabled targeted integration of over 5 kb DNA (Anzalone et al., Nat Biotechnol, 40(5), 731-740, 2022; Yarnall et al., Nat Biotechnol. 2022 Nov. 24). This technology expands the capabilities of precise genome editing by allowing large, multiplexed gene insertion without the need of double-stranded breaks (DSBs).

Although integrase-mediated genome engineering is a powerful tool for editing DNA, it is not without limitations. Low efficiency is among one of the limitations associated with this technology, meaning that not aall cells are successfully edited. This can limit the usefulness of the technique, especially when working with difficult-to-manipulate cells or tissues. One direction to improve the efficiency is to provide higher amount of cargo DNA substrate. However, site-specific recombinases require circular double-stranded DNA (dsDNA), usually a plasmid, as the transgene donor. The use of dsDNA suffers from high cytotoxicity (especially at higher doses) when delivered into cells causing significant cell death, mainly due to cellular innate immune responses mediated by cytosolic DNA sensing pathways, such as Toll-like receptor 9 (TLR9), cyclic GMP-AMP synthase (cGAS), or stimulator of interferon genes (STING) (Briard et al., Physiology (Bethesda), 35(2), 112-124, 2020; Schlee & Hartmann, Nat Rev Immunol, 16(9), 566-580, 2016; Zahid et al., Front Immunol, 11, 613039, 2020). Low cell viability directly impacts the efficiency of gene editing, thus limits the application of SSRs in gene therapy.

A technology platform purifying circular single-stranded DNA (cssDNA) carrying up to 20 kb of gene payload from engineered phagemids in a clean and scalable fashion was developed. It was established that cssDNA homology-directed repair (HDR) donor templates enable higher genome engineering efficiency. More importantly, when delivered into cells, cssDNA does not trigger cellular innate immune response, thus has decreased cytotoxicity when compared to dsDNA template counterpart, demonstrating its potential application for genetic disorders and immune cell therapy (Xie et al., 2022). Moreover, because of the single-stranded structure, cssDNA possesses only half of the molecular weight of its double-stranded DNA counterpart. These intrinsic properties made it possible to achieve higher molar mass of cssDNA using same amount of material in genetic engineering for higher efficiency.

cssDNA has been demonstrated to be a superior donor template for HDR-mediated gene knock-in, it was reasoned that it could also be suitable for wider applications, such as serving as DNA substrate for SSR. The cssDNA might not be an optimal substrate of SSR, as the recognition sites (attP and attB) for SSR are considered double-stranded. Therefore, the attP or attB site in cssDNA could need to be hybridized with a complementary oligo to form double-stranded site for SSR recognition. It was hypothesized that a hybrid DNA (cssDNA with partial double-stranded at integrase recognition site) is a suitable substrate for SSR mediated engineering. In the case of the “landing pad” (e. g., attB site) is present in the genome, integrase is expected to “cut” and “paste” transgene payload in the cssDNA molecule into the host genome, the cell's own repair mechanism will synthesize the second strand to restore the double helical structure of the genomic DNA, thus completing the permanent gene transfer. Due to the limited double-stranded region (only <50 bp) in the hybrid DNA, its cellular toxicity is expected to be low, comparing to fully duplexed dsDNA.

### Example 2: CssDNA with Duplexed Region at attB and attP Sites is an Efficient DNA Substrate for Bxb1 Integrase

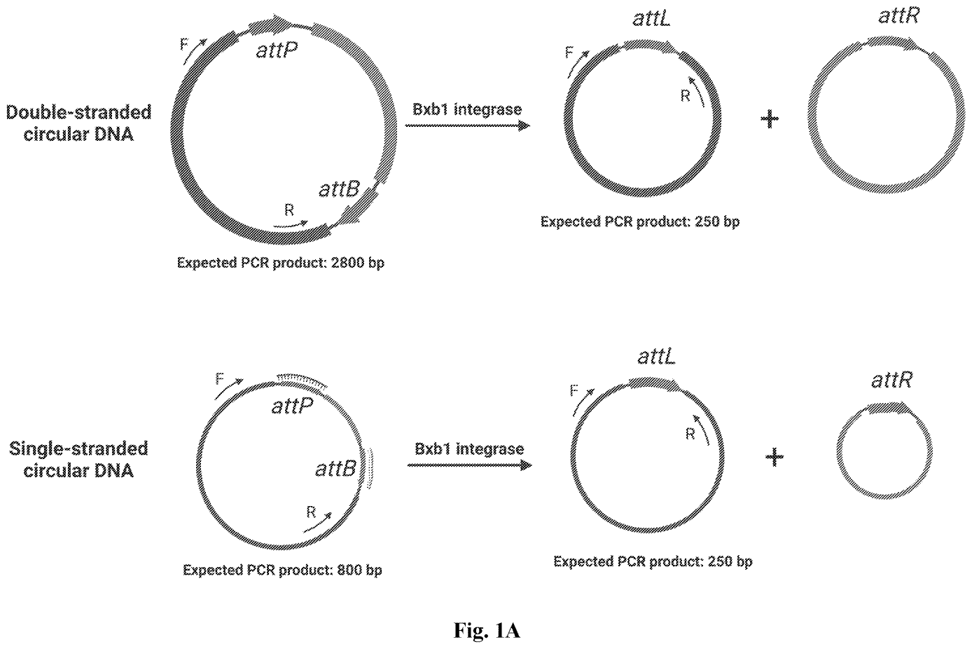

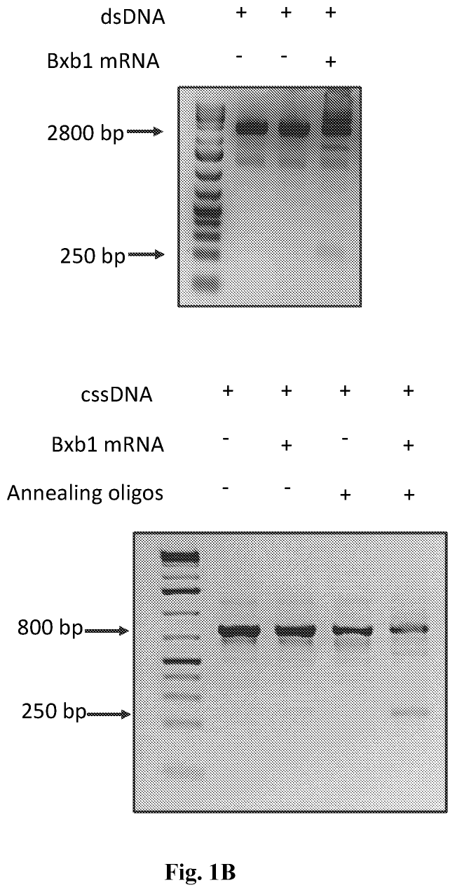

The present study first designed a double-stranded phagemid with payload flanked by attB and attP sites and used this phagemid to manufacture cssDNA. The single stranded payload with two attB and attP sites were maintained while the antibiotic resistant gene and E. coli replication origin regions were removed in purified cssDNA. Partially duplexed cssDNA were prepared by annealing cssDNA with two complementary annealing oligos of attP and attB sites. Bxb1 mRNA codon-optimized for human cell expression were synthesized by in vitro transcription method. Bxb1 is expected to catalyze integrative recombination between attP and attB site to produce attL and attR, resulting in two minicircles. The forward and reverse primers were designed in outward direction. Using this design, the amplification product is expected to be 2800 bp and 800 bp for un-recombined dsDNA and cssDNA (FIG. 1A). Once the attP and attB recombination happens, the forward and reverse primers will amplify a 250 bp band from the resulting minicircle containing attL site (FIG. 1A). The circular DNAs (dsDNA, cssDNA or partially duplexed cssDNA) were co-electroporated with Bxb1 mRNA to K562 cells. 24 hours after electroporation, total DNA were isolated and the existence of minicircle were determined by PCR methods. As expected, when double-stranded phagemid DNA containing attB and attP sites were co-electroporated with Bxb1 mRNA, PCR amplification resulted in a 2800 bp band and a 250 bp band. However, without Bxb1 integrase, only 2800 bp band could be detected (FIG. 1). When cssDNA was electroporated into K562 cells, only 800 bp product was amplified by PCR. When cssDNA was co-electroporated with Bxb1 integrase, in addition to the 800 bp band, a noticeable but faint 250 bp PCR product was also detected. When the hybrid DNA (cssDNA with duplex at attP and attB site) were co-electroporated with Bxb1 integrase, the 250 bp PCR product was much stronger, indicating much more minicircle products were produced by attB×attP recombination. As a negative control, the hybrid DNA in the absence of Bxb1 integrase did not result in 250 bp PCR band (FIG. 1). These results demonstrated both cssDNA alone or cssDNA with partially duplexed region attP and attB sites is an efficient DNA substrate for Bxb1 integrase mediated recombination.

### Example 3: Efficient Genome Integration in iPSC Cells Using Integrase and Partially Duplexed cssDNA Substrate

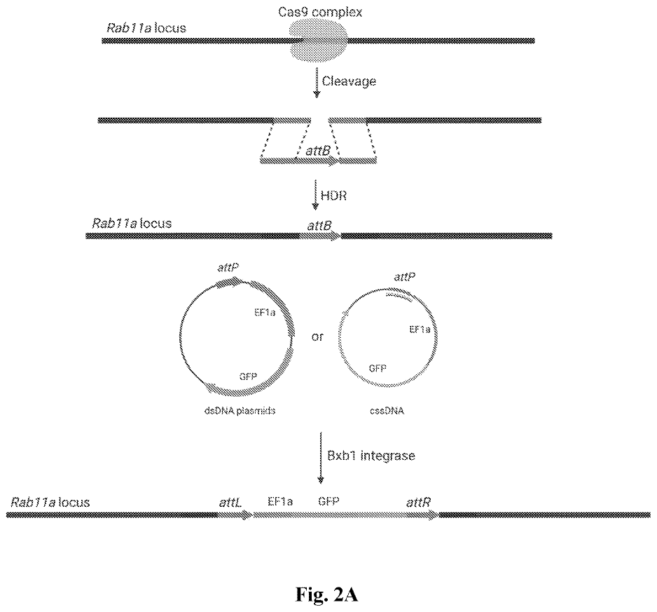

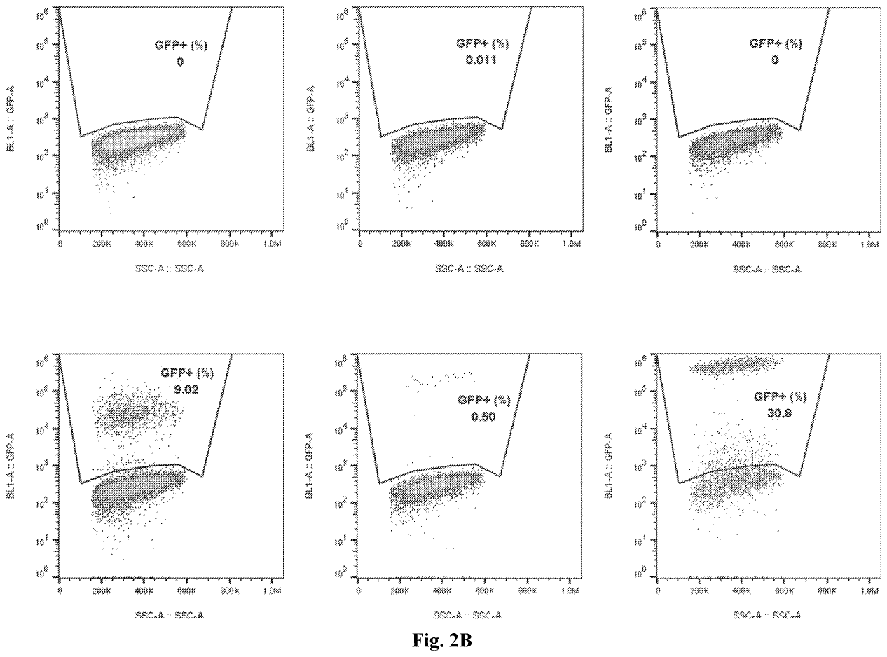

The present study next sought to determine if cssDNA or its partially duplexed form can be used as integrase DNA substrate for human cell genome engineering. The present study intended to knock in the “landing pad” (attB site) in Rab11a locus in human induced Pluripotent Stem Cells (iPSCs). It was demonstrated a high cutting efficiency Rab11a gRNA, which was also used for targeted knock-in at high efficiency in multiple human cells. Ribonucleoprotein (RNP) targeting Rab11a locus along with an Rab11a-attB oligo (46 base of attB flanked by 50 bases of homology arm from each side) were used for targeted attB landing pad knock-in (FIG. 2A). Phagemid with EF1a promoter driven GFP payload downstream of attP site were cloned and used for cssDNA manufacturing (FIG. 2A). cssDNA was either used directly or to from a partially duplexed cssDNA by annealing with a complementary attB oligo. Cultured iPSCs were co-electroporated with Rab11a-RNP, Rab11a-attB oligo, Bxb1 mRNA together with cargo DNA substrate in different forms (dsDNA, cssDNA or hybrid DNA). 15 days post electroporation, iPSCs with stable GFP expression were determined by flow cytometry. Cells in all conditions were treated with Rab11a RNP and Rab11a-attB oligo. As shown in FIG. 2B, there were no GFP positive cells in the absence of Bxb1 integrase. With Bxb1 integrase, dsDNA substrate resulted in ~9% of GFP positive cells, cssDNA substrate resulted in less than 1% GFP positive cells. However, when partially duplexed cssDNA substrate was used, GFP cargo was integrated into the genome of ~30% of cells. These data demonstrate that the partially duplexed form of cssDNA containing double-stranded region only at the integrase recognition site (attP) is a superior DNA substrate for efficient integrase-mediated cargo DNA integration.

To the best knowledge of the inventors, this is the first time cssDNA or its partially duplexed form as integrase substrate for genome engineering with SSR has been demonstrated. The advantages of cssDNA (e.g., lower cellular toxicity, higher molecular mass efficiency and genetic engineering efficiency, etc.) can be fully applied to additional applications in genome engineering in a homology independent manner.

### Example 4: Efficient Genome Integration in K562 Cells Using Transposase and cssDNA Donor

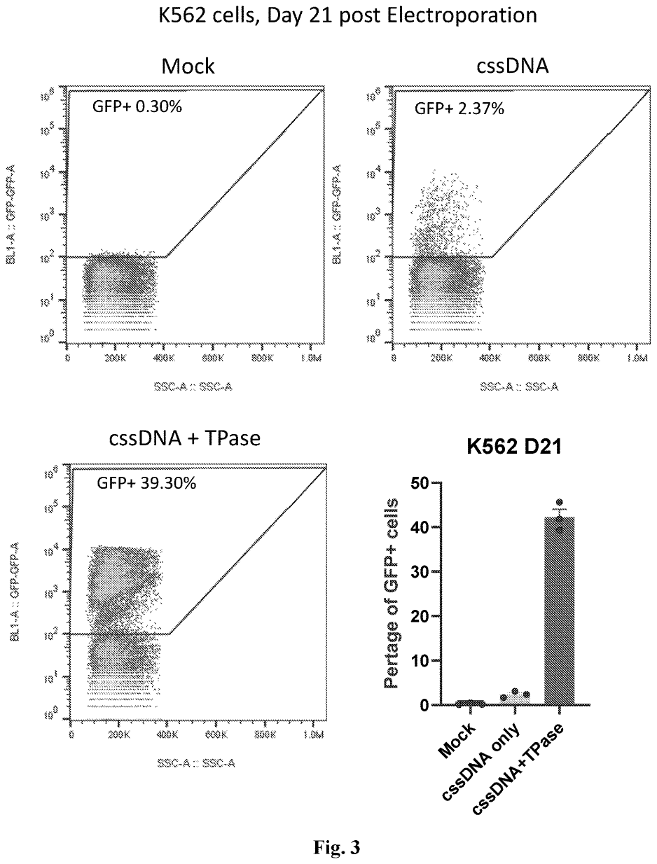

The present study sought to determine if cssDNA can be used as DNA donor substrate for human cell genome engineering by transposase system. The present study first designed a double-stranded phagemid encoding EF1a-GFP reporter payload flanked by ITR sites and used this phagemid to manufacture cssDNA donor template. The single stranded payload with two ITR sites were maintained while the antibiotic resistant gene and E. coli replication origin regions were removed in purified cssDNA. Codon-optimized NLS tagged PiggyBac Transposase mRNA for human cell expression were synthesized by in vitro transcription method. PiggyBac Transposase is expected to recognize conventional dsDNA form of the 5′ and 3′ ITR sites and catalyze integration of the DNA sequences flanked by the ITR sites. Once the stable integration happens, the GFP reporter expression can be detected for over a few weeks. In the study, the circular DNAs containing the EF1a-GFP flanked by 5′ and 3′ ITRs were co-electroporated with NLS tagged PiggyBac Transposase mRNA to K562 cells. At day 21 post electroporation, ~39.30% of GFP reporter expression was observed from stably integrated EF1a-GFP transgene, with only ~2.37% of GFP expression from the cssDNA donor template alone group, indicating substantial stable genomic integration of the reporter transgene by NLS tagged PiggyBac Transposase. (FIG. 3)

### Example 5: Efficient Genome Integration in Human Primary T Cells Using Transposase and cssDNA Donor

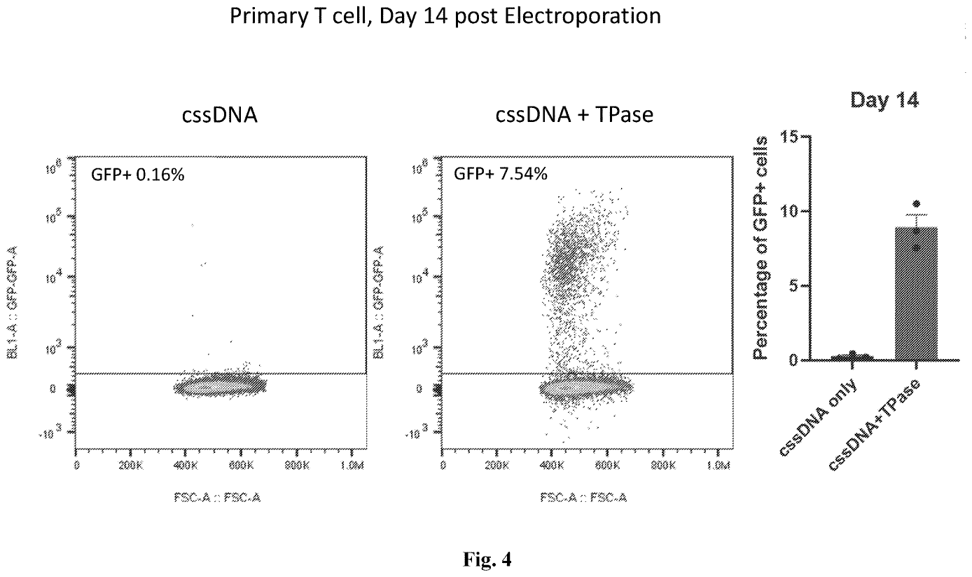

The present study then sought to determine if transpose mediated genomic integration with cssDNA donor template is applicable in primary cell types. The present study used the same cssDNA EF1a-GFP reporter payload flanked by 5′ and 3′ ITR sites. In the study, the circular DNAs containing the EF1a-GFP flanked by 5′ and 3′ ITRs were co-electroporated with NLS tagged PiggyBac Transposase mRNA to human peripheral blood derived CD4/CD8 double positive T lymphocytes. At day 14 post electroporation, ~7.54% of GFP reporter expression was observed from stably integrated EF1a-GFP transgene, with only ~0.16% of GFP expression from the cssDNA donor template alone group, indicating substantial stable genomic integration of the reporter transgene by NLS tagged PiggyBac Transposase. (FIG. 4). To the best knowledge of the inventors, this is the first time cssDNA as transposase donor DNA substrate for genome engineering. The advantages of cssDNA (e.g., lower cellular toxicity, higher molecular mass efficiency and genetic engineering efficiency, etc.) can be fully applied to additional applications in genome engineering in a homology independent manner.

### Example 6: Materials and Methods

Generation of Template Circular Single-Stranded DNA from M13 Phage

Donor template sequences for integrase system or Transposase are constructed as dsDNA and cloned into phagemid vector. An XL1-Blue E. coli strain was co-transformed with the M13 helper plasmid and phagemid containing double-stranded donor template and selected on agar plates with kanamycin (50 μg/mL) and carbenicillin (100 μg/mL). A single colony was selected and grown for −24 hours (37° C., 225 rpm) in 250 mL 2×YT media (1.6% tryptone, 1% yeast extract, 0.25% NaCl) to reach OD₆₀₀ between 2.5-3.0. The bacteria were pelleted by centrifugation and the phage particles in the supernatant were precipitated with PEG-8000. The precipitated phage particles were then pelleted by centrifugation, washed, and lysed in 20 mM MOPS, 1M Guanidine-HCl and 2% Triton X-100. The cssDNA released from the phage were then extracted with NucleoBond Xtra Midi EF kit (Macherey-Nagel) following the manufacturer's instructions. The concentration of cssDNA was determined by Nanodrop for ssDNA and the yields were 10 μg per ml of liquid culture. Ratios of Absorbance (A₂₆₀ nm/A₂₈₀ nm and 260 nm/230 nm) reflect consistent purity (1.8 and >2, respectively) from serial preps. Recombinant cssDNA is verified by Sanger DNA sequencing using custom-designed staggered sequencing primers for complete coverage.

### Cell Culture

K562 (ATCC) cells were maintained in RPMI-1640 media with 10% FBS and 1% penicillin and streptomycin. iPSCs (ThermoFisher Scientific) were cultured in complete StemFlex (ThermoFisher Scientific) media in vitronectin-coated flasks. iPSC colonies were checked regularly and passaged using ReLeSR (StemCell Technologies) every 3-4 days of culture. iPSCs were ready for electroporation after 2-3 passages. All cells were maintained in a humidified incubator at 37° C. and 5% C02, unless otherwise specified. Cell count viability was determined using a Via2-Cassette in NucleoCounter® NC-202 (ChemoMetec) on specified days after engineering.

Partial Duplexed cssDNA Preparation

To prepare partial duplexed cssDNA at attP and attB sites, reverse complementary attP and attB oligos (50 pmol each) were mixed with cssDNA (25 μmol) in 1× buffer NEB buffer r2.1. The mixture were incubated at 75° C. temperature for 3 minutes followed by colling to room temperature at 0.1° C./see ramp rate. The oligo annealed cssDNA can be stored in 4° C. for future use.

### Electroporation

K562, iPSC and primary T cell electroporation were performed using the Amaxa™ 96-well Shuttle™ with the 4D Nucleofector (Lonza). 25 picomole of sNLS-SpCas9-sNLS Nuclease (Aldevron) along with 50 pmol of sgRNA (synthesized at Integrated DNA Technologies) were used per reaction. Cas9 nucleases and sgRNAs were precomplexed in supplemented Nucleofector® Solution for 20 min at room temperature and the RNP solution was increased to a final volume of 2.5 μL (10×) per electroporation reaction. For mRNA delivery nucleases, 1 μg of Bxb1 mRNA was co-electroplated with 50 pmol of sgRNA and indicated amount of dsDNA or cssDNA. For electroporation of PiggyBac Transposase, 2 ug mRNA is used with 2 μg of cssDNA. For electroporating K562 cells, an SF Cell Line 4D-Nucleofector™ Kit and 250,000-500,000 cells per reaction were used. For electroporating iPSC cells, 100,000 cells per reaction were used with a P3 Primary Cell 4D-Nucleofector™ Kit. After electroporation, the cells were placed in a humidified 32° C. incubator with 5% C02 for 12-24 hours, followed by transferring to 37° C. incubator for 3-10 additional days. For electroporating primary human T lymphocyte cells, Primary T cells were isolated and enriched from Leukopak using Pan T Cell MicroBead Cocktail with MultiMCAS Cell24 Separator Plus (Miltenyi), CD4+ and CD8+ pan T cells were cryopreserved for later use. T cells were cultured and expanded in TexMACS Medium (Miltenyi) supplemented with 200 IU/mL Human IL-2 IS (Miltenyi). T cells were activated for 2 days with T Cell TransAct (Miltenyi) before electroporation. 48 h after initiating T-cell initiation and activation, T cells were electroporated using Amaxa™ 96-well Shuttle™ in 4D Nucleofector. 2×10⁶ cells were mixed with 2 μg of PiggyBac Transposase in vitro transcribed mRNA and 2 ug ITR-EF1a-GFP-ITR cssDNA. Following electroporation, cells were incubated at 32° C., 5% C02 for 24 hours. Cells were then washed and subsequently transferred into G-Rex 24 Multi-Well Cell Culture Plate (Wilson Wolf) in standard culture conditions at 37° C., 5% C02 in IL-2 supplemented TexMACS medium and replenished every 3-4 days before GFP reporter analysis at indicated days after electroporation.

Bxb1 mRNA Preparation by In Vitro Transcription

Codon-optimized Bxb1 for human cell expression fused with SV40 nucleic localization signal on N terminal were cloned into pGEM-4A vector. One μg of linearized DNA template was in vitro transcribed in a 20 μl reaction using 1.5 μl T7 RNA polymerase mix and 5 mM each of GTP, ATP, CTP, and UTP from the HiScribe T7 mRNA Synthesis Kit (NEB E2080S) following manufacturer's instructions. RNA was purified with Zymo min-spin column, followed by elution in 80 μL nuclease-free water.

PiggyBac Transposase mRNA Preparation by In Vitro Transcription

Codon-optimized PiggyBac Transposase for human cell expression were cloned into pGEM-4A vector. One μg of linearized DNA template was in vitro transcribed in a 20 μl reaction using 1.5 μl T7 RNA polymerase mix and 5 mM each of GTP, ATP, CTP, and UTP from the HiScribe T7 mRNA Synthesis Kit (NEB E2080S) following manufacturer's instructions. RNA was purified with Zymo min-spin column, followed by elution in 80 μL nuclease-free water.

### K562 Cell DNA Extraction and PCR Amplification

K562 were electroporated with circular DNA constructs (dsDNA, cssDNA or partially duplexed cssDNA) with or without Bxb1 mRNA. Total DNAs were extracted 24 hours post electroporation by Monarch Genomic DNA Purification Kit (New England BioLabs) following manufacturer's instructions. The existence of minicircle after attB×attP recombination were detected by PCR amplification using the forward and reverse primer.

### Flow Cytometry Analysis

All flow cytometry was performed on an Attune NxT flow cytometer with a 96-well autosampler (ThermoFisher Scientific). Unless otherwise indicated, cells were collected 4-7 days post electroporation, resuspended in fluorescence-activated cell sorting (FACS) buffer (2% FBS in PBS) and stained with 7-AAD (BioLegend), and the indicated cell-surface marker. To obtain comparable live cell counts between conditions, events were recorded from an equivalent fixed volume for all samples. Data analysis was performed using FlowJo_v10.8.0_CL software with exclusion of subcellular debris, singlet gating and live:dead stain. Analyzed graphs were produced with Prism 9 (GraphPad).

### DNA RNA Sequence Information Used in the Present Study

The DNA and RNA sequences used in the present study are listed in Table 1 below:

TABLE 1

| ID | 5′ to 3′ sequence |
|---|---|
| attP site | GTGGTTTGTCTGGTCAACCACCGCGgtCTCAGTGGTGTACGGT |
| | ACAAACCCA (SEQ ID NO: 1) |
| attB site | GGCCGGCTTGTCGACGACGGCGgtCTCCGTCGTCAGGATCATC |
| | CGG (SEQ ID NO: 2) |
| attP reverse | TGGGTTTGTACCGTACACCACTGAGacCGCGGTGGTTGACCA |
| complementary oligo | GACAAACCAC (SEQ ID NO: 3) |
| attB reverse | CCGGATGATCCTGACGACGGAGacCGCCGTCGTCGACAAGCC |
| complementary oligo | GGCC (SEQ ID NO: 4) |
| Rab11a gRNA | GGUAGUCGUACUCGUCGUCG (SEQ ID NO: 5) |
| Forward PCR primer | CAGCCCTTCCTTTTTGTGCG (SEQ ID NO: 6) |
| Reverse PCR primer | GGGGAAACTGAGTCCTGGAA (SEQ ID NO: 7) |
| ITR-EF1a-GFP-ITR | TTAACCCTAGAAAGATAGTCTGCGTAAAATTGACGCATGCATT |
| | CTTGgctccggtgcccgtcagtgggcagagcgcacatcgccca |
| | cagtccccgagaagttggggggaggggtcggcaattgaaccgg |
| | tgcctagagaaggtggcgcggggtaaactgggaaagtgatgtc |
| | gtgtactggctccgcctttttcccgaggggggggagaaccgta |
| | tataagtgcagtagtcgccgtgaacgttctttttcgcaacggg |
| | tttgccgccagaacacaggtaagtgccgtgtgtggttcccgcg |
| | ggcctggcctctttacgggttatggcccttgcgtgccttgaat |
| | tacttccacgcccctggctgcagtacgtgattcttgatcccga |
| | gcttcgggttggaagtgggtgggagagttcgaggccttgcgct |
| | taaggagccccttcgcctcgtgcttgagttgaggcttggcctg |
| | ggcgctggggccgccgcgtgcgaatctggtggcaccttcgcgc |
| | ctgtctcgctgctttcgataagtctctagccatttaaaatttt |
| | tgatgacctgctgcgacgctttttttctggcaagatagtcttg |
| | taaatgcgggccaagatctgcacactggtatttcggtttttgg |
| | ggccgcgggcggcgacggggcccgtgcgtcccagcgcacatgt |
| | tcggcgaggcggggcctgcgagcgcggccaccgagaatcggac |
| | gggggtagtctcaagctggccggcctgctctggtgcctggcct |
| | cgcgccgccgtgtatcgccccgccctgggcggcaaggctggcc |
| | cggtcggcaccagttgcgtgagcggaaagatggccgcttcccg |
| | gccctgctgcagggagctcaaaatggaggacgcggcgctcggg |
| | agagcgggcgggtgagtcacccacacaaaggaaaagggccttt |
| | ccgtcctcagccgtcgcttcatgtgactccacggagtaccggg |
| | cgccgtccaggcacctcgattagttctcgagcttttggagtac |
| | gtcgtctttaggttggggggaggggttttatgcgatggagttt |
| | ccccatactgagtgggtggagactgaagttaggccagcttggc |
| | acttgatgtaattctccttggaatttgccctttttgagtttgg |
| | atcttggttcattctcaagcctcagacagtggttcaaagtttt |
| | tttcttccatttaaggtgtcgtgaacgcgtattaatacgctgc |
| | tagcggtaccGCCACCATGGGATCGGGTGGGACTAGTGGCAGC |
| | AAGGGCGAGGAGCTGTTCACCGGGGTGGTGCCCATCCTGGTCG |
| | AGCTGGACGGCGACGTAAACGGCCACAAGTTCAGCGTGCGCGG |
| | CGAGGGCGAGGGCGATGCCACCAACGGCAAGCTGACCCTGAAG |
| | TTCATCTGCACCACCGGCAAGCTGCCCGTGCCCTGGCCCACCC |
| | TCGTGACCACCCTGACCTACGGCGTGCAGTGCTTCAGCCGCTA |
| | CCCCGACCACATGAAGCGCCACGACTTCTTCAAGTCCGCCATG |
| | CCCGAAGGCTACGTCCAGGAGCGCACCATCAGCTTCAAGGACG |
| | ACGGCACCTACAAGACCCGCGCCGAGGTGAAGTTCGAGGGCGA |
| | CACCCTGGTGAACCGCATCGAGCTGAAGGGCATCGACTTCAAG |
| | GAGGACGGCAACATCCTGGGGCACAAGCTGGAGTACAACTTCA |
| | ACAGCCACAACGTCTATATCACCGCCGACAAGCAGAAGAACGG |
| | CATCAAGGCCAACTTCAAGATCCGCCACAACGTGGAGGACGGC |
| | AGCGTGCAGCTCGCCGACCACTACCAGCAGAACACCCCCATCG |
| | GCGACGGCCCCGTGCTGCTGCCCGACAACCACTACCTGAGCAC |
| | CCAGTCCGTGCTGAGCAAAGACCCCAACGAGAAGCGCGATCAC |
| | ATGGTCCTgCTGGAgTTCGTGACCGCCGCCGGGATCACTggaa |
| | ccggtGCTggaagtggtTGATAAgtttaaacccgctgatcagc |
| | ctcgactgtgccttctagttgccagccatctgttgtttgcccc |
| | tcccccgtgccttccttgaccctggaaggtgccactcccactg |
| | tcctttcctaataaaatgaggaaattgcatcgcattgtctgag |
| | taggtgtcattctattctggggggggggtggggcaggacagca |
| | agggggaggattgggaagacaatagcaggcatgctggggatgc |
| | ggtgggctctatggCAGACCGATAAAACACATGCGTCAATTTT |
| | ACGCATGATTATCTTTAACGTACGTCACAATATGATTATCTTT |
| | CTAGGGTTAA |
| | (SEQ ID NO: 8) |
| PiggyBac Transposase | atggccccaaaaaagaaaagaaaagtgtatccctatgatgtcc |
| | ccgattatgccggttcaggctctagcctggacgacgagcacat |
| | cctgagcgccctgctgcagagcgacgacgaactggtgggcgag |
| | gacagcgacagcgaggtcagcgaccacgtgtccgaggacgacg |
| | tgcagtccgacaccgaggaagccttcatcgacgaggtgcacga |
| | agtgcagcctaccagcagcggctccgagatcctggacgagcag |
| | aacgtgatcgagcagcctggcagctccctggccagcaacagaa |
| | tcctgaccctgccccagagaaccatcagaggcaagaacaagca |
| | ctgctggtccacctccaagagcaccaggcggagcagagtgtcc |
| | gccctgaacatcgtgcggagccagaggggccccaccagaatgt |
| | gcagaaacatctacgaccccctgctgtgcttcaagctgttctt |
| | caccgacgagatcatcagcgagatcgtgaagtggaccaacgcc |
| | gagatcagcctgaagaggcgggagagcatgaccagcgccacct |
| | tcagagacaccaacgaggacgagatctacgccttcttcggcat |
| | cctggtgatgaccgccgtgagaaaggacaaccacatgagcacc |
| | gacgacctgttcgacagatccctgagcatggtgtacgtgtccg |
| | tgatgagcagagacagattcgacttcctgatcagatgcctgag |
| | aatggacgacaagagcatcagacccaccctgcgggagaacgac |
| | gtgttcacccccgtgcggaagatctgggacctgttcatccacc |
| | agtgcatccagaactacacccctggcgcccacctgaccatcga |
| | tgagcagctgctgggcttcagaggcagatgccccttcagagtg |
| | tacatccccaacaagcccagcaagtacggcatcaagatcctga |
| | tgatgtgcgacagcggcaccaagtacatgatcaacggcatgcc |
| | ctacctgggcagaggcacccagacaaacggcgtgcccctgggc |
| | gagtactacgtgaaagaactgagcaagcctgtgcatggcagct |
| | gcaggaacatcacctgcgacaactggttcaccagcatccccct |
| | ggccaagaacctgctgcaggaaccctacaagctgaccatcgtg |
| | ggcaccgtgcggagcaacaagcgggagatcccagaggtgctga |
| | agaacagcagatccagacctgtgggaacaagcatgttctgctt |
| | cgacggccccctgaccctggtgtcctacaagcccaagcccgcc |
| | aagatggtgtacctgctgtccagctgcgacgaggacgccagca |
| | tcaacgagagcaccggcaagccccagatggtgatgtactacaa |
| | ccagaccaagggcggcgtggacaccctggaccagatgtgcagc |
| | gtgatgacctgcagcagaaagaccaacagatggcccatggccc |
| | tgctgtacggcatgatcaatatcgcctgcatcaacagcttcat |
| | catctacagccacaacgtgtccagcaagggcgagaaggtgcag |
| | agccggaagaaattcatgcggaacctgtacatgagcctgacct |
| | ccagcttcatgagaaagagactggaagcccccaccctgaagag |
| | atacctgcgggacaacatcagcaacatcctgcccaaggaagtg |
| | ccaggaacaagcgacgacagcaccgaggaacccgtgatgaaga |
| | agaggacctactgcacctactgtcccagcaagatcagaagaaa |
| | ggccaacgccagctgcaagaaatgcaaaaaagtgatctgccgg |
| | gagcacaacatcgacatgtgccagagctgtttctgagaattcc |
| | tagagctcgctgatcagcctcgactgtgccttctagttgccag |
| | ccatctgttgtttgcccctcccccgtgccttccttgaccctgg |
| | aaggtgccactcccactgtcctttcctaataaaatgaggaaat |
| | tgcatcgcattgtctgagtaggtgtcattctattctggggggt |
| | ggggggggcaggacagcaagggggaggattgggaagagaatag |
| | caggcatgctgggga |
| | (SEQ ID NO: 9) |

### ENUMERATED EMBODIMENTS

In some aspects, the present invention is directed to the following non-limiting embodiments:

Embodiment 1: A circular single-stranded DNA (cssDNA) molecule comprising a recombinase recognition sequence recognized by a recombinase.

Embodiment 2: The cssDNA molecule of Embodiment 1, wherein the recombinase is a site-specific recombinase (SSR).

Embodiment 3: The cssDNA molecule of Embodiment 2, wherein the site-specific recombinase is a tyrosine integrase, a serine integrase, or a site-specific transposase.

Embodiment 4: The cssDNA molecule of Embodiment 3, wherein the recombinase recognition sequence is a locus of X-over P1 (loxP) sequence, a flippase recognition target (FRT) sequence, a rox sequence, an attachment-phage (attP) sequence, an attachment-bacteria (attB) sequence, an attachment-left (attL) sequence, an attachment-right (attR) sequence, a VloxP sequence, a vox sequence, or a transposase recognition inverted terminal repeat (ITR).

Embodiment 5: The cssDNA molecule of anyone of Embodiments 1-4, further comprising a payload sequence.

Embodiment 6: The cssDNA molecule of Embodiment 5, wherein the payload sequence encodes an RNA molecule or a polypeptide.

Embodiment 7: The cssDNA molecule of any one of Embodiments 1-6, wherein the payload sequence comprises a length of about 1 nucleotide to about 50,000 nucleotides.

Embodiment 8: The cssDNA molecule of any one of Embodiments 1-7, wherein the cssDNA molecule is purified or enriched.

Embodiment 9: A double stranded DNA (dsDNA) molecule comprising: a template sequence encoding a cssDNA comprising a recombinase recognition sequence recognized by a recombinase; and a cssDNA conversion sequence.

Embodiment 10: The dsDNA molecule of Embodiment 9, wherein the double stranded DNA molecule is a linear dsDNA molecule or a circular dsDNA molecule.

Embodiment 11: The dsDNA molecule of any one of Embodiments 9-10, wherein the cssDNA conversion sequence is a sequence originated or derived from an M13 phage f1 origin, an M13 phage replication initiator, an M13 phage replication terminator, or an M13 phage packing signal (PS).

Embodiment 12: The dsDNA molecule of any one of Embodiments 9-11, further comprising a payload sequence, or a payload sequence insertion site for inserting the payload sequence.

Embodiment 13: The dsDNA molecule of Embodiment 12, wherein the payload insertion site is a multiple cloning site.

Embodiment 14: The dsDNA molecule of any one of Embodiments 9-13, which is one single DNA molecule, or two or more separate DNA molecules.

Embodiment 15: A kit comprising: the cssDNA molecule of any one of Embodiments 1-8, or the dsDNA molecule of any one of Embodiments 9-14 and a system that converts the dsDNA molecule to the cssDNA; and a recombinase or a nucleic acid encoding the recombinase, wherein the recombinase recognizes the recombinase recognition sequence of the cssDNA.

Embodiment 16: The kit of Embodiment 15, wherein the recombinase is a serine integrase, a tyrosine integrase, or a site-specific transposase.

Embodiment 17: The kit of Embodiment 15, wherein the recombinase is Cre, Flp, Dre, SCre, VCre, Vika, B2, B3, KD, ΦDC31, Bxb1, RDF, λ, HK022, HP1, γδ, ParA, Gin, R4, TP901-1, TG1, PhiRv1, PhiBT1, SprA, XisF, TnpX, R, A118, spoIVCA, MR11, SCCmec, TndX, XerC, XerD, XisA, Hin, Cin, mrpA, beta, PhiFC1, Fre, Clp, sTre, FimE, HbiF, φFC1, φC1, q 370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), Minos, R1, R2, R3, R4, R5, Mu transposase, Tn3 transposase, Tn5 transposase, Tn7 transposase, Tol2, Sleeping Beauty transposase, or PiggyBac transposase.

Embodiment 18: The kit of any one of Embodiments 15-17, wherein kit comprises the nucleic acid encoding the recombinase, wherein the nucleic acid encoding the recombinase is an mRNA, a single stranded DNA, or a double stranded DNA.

Embodiment 19: The kit of any one of Embodiments 15-18, further comprising an oligonucleotide fully or partially complementary with the cssDNA molecule.

Embodiment 20: The kit of Embodiment 19, wherein the oligonucleotide is a primer that converts the cssDNA molecule to a circular double stranded DNA molecule inside a cell.

Embodiment 21: The kit of any one of Embodiments 19-20, wherein the oligonucleotide comprises a length of about 5 nucleotides to about 200 nucleotides.

Embodiment 22: The kit of any one of Embodiments 15-21, wherein the kit comprises the nucleic acid encoding the recombinase, and wherein the nucleic acid encoding the recombinase is part of the cssDNA molecule or the dsDNA molecule.

Embodiment 23: The kit of any one of Embodiments 15-22, further comprising a component for introducing the cssDNA, and the recombinase or the nucleic acid encoding the recombinase, into the cell.

Embodiment 24: A system comprising: the kit of any one of Embodiments 15-23; and a cell to be engineered.

Embodiment 25: The system of Embodiment 24, wherein a genomic DNA or a mitochondrial DNA of the cell comprises a landing pad sequence for the integration of the cssDNA molecule into the genomic DNA or the mitochondrial DNA.

Embodiment 26: The system of Embodiment 25, wherein the landing pad sequence is recognized by the recombinase such that the recombinase opens up the cssDNA molecule and inserts the linearized cssDNA molecule into the genomic DNA or the mitochondrial DNA using the landing pad sequence as an anchor site.

Embodiment 27: The system of any one of Embodiments 25-26, wherein the landing pad sequence is a locus of X-over P1 (loxP) sequence, a flippase recognition target (FRT) sequence, a rox sequence, an attachment-phage (attP) sequence, an attachment-bacteria (attB) sequence, an attachment-left (attL) sequence, an attachment-right (attR) sequence, a VloxP sequence, a vox sequence, or a transposase recognition inverted terminal repeat (ITR).

Embodiment 28: The system of any one of Embodiments 24-27, wherein the cell is a bacterial cell, a plant cell, or a mammalian cell.

Embodiment 29: The system of any one of Embodiments 24-28, wherein the cell is an isolated cell, a cell from a cell line, a primary cell, or a cell in a subject Embodiment 30: The system of any one of Embodiments 24-29, wherein the cell is a pluripotent stem cell, an induced pluripotent stem cell (iPSCs), a hematopoietic stem cell (HSCs), a B-cell, a T-cell, a natural killer (NK) cell, or a terminally differentiated non-dividing cell, such as a neuron, a muscle, or a hepatocyte.

Embodiment 31: A method for performing genetic engineering in a cell, comprising introducing into the cell:
the cssDNA of any one of Embodiments 1-8; and
a recombinase or a nucleic acid encoding the recombinase,
wherein the recombinase recognizes the recombinase recognition sequence of the cssDNA to integrate the cssDNA into a genomic DNA or a mitochondrial DNA of the cell.

Embodiment 32: The method of Embodiment 31, wherein the recombinase is a serine recombinase, a tyrosine recombinase, or a site-specific transposase.

Embodiment 33: The method of Embodiment 31, wherein the recombinase is Cre, Flp, Dre, SCre, VCre, Vika, B2, B3, KD, ΦDC31, Bxb1, RDF, λ, HK022, HP1, γδ, ParA, Gin, R4, TP901-1, TG1, PhiRv1, PhiBT1, SprA, XisF, TnpX, R, A118, spoIVCA, MR11, SCCmec, TndX, XerC, XerD, XisA, Hin, Cin, mrpA, beta, PhiFC1, Fre, Clp, sTre, FimE, HbiF, φFC1, φC1, q 370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), Minos, R1, R2, R3, R4, R5, Mu transposase, Tn3 transposase, Tn5 transposase, Tn7 transposase, Tol2, Sleeping Beauty transposase, or PiggyBac transposase.

Embodiment 34: The method of any one of Embodiments 31-33, further comprising introducing into the cell an oligonucleotide fully or partially complementary to at least a portion of the cssDNA molecule.

Embodiment 35: The method of Embodiment 34, wherein a cellular machinery of the cell converts the cssDNA molecule into a circular double stranded DNA molecule using the oligonucleotide as a primer.

Embodiment 36: The method of any one of Embodiments 34-35, wherein the oligonucleotide comprises a length of about 5 nucleotides to about 200 nucleotides.

Embodiment 37: The method of any one of Embodiments 31-36, which comprises introducing into cells a sense stranded cssDNA, an antisense stranded cssDNA, or a mixture of both.

Embodiment 38: The method of Embodiments 31-37, wherein the genomic DNA or the mitochondrial DNA of the cell comprises a landing pad sequence for the integration of the cssDNA molecule into the genomic DNA or the mitochondrial DNA.

Embodiment 39: The method of Embodiment 38, further comprising culturing the cell under a condition sufficient for the landing pad sequence to be recognized by the recombinase and for the recombinase to open up the cssDNA molecule and insert the linearized cssDNA molecule into the genomic DNA or the mitochondrial DNA using the landing pad sequence as an anchor site.

Embodiment 40: The method of any one of Embodiments 31-39, wherein the landing pad sequence is a locus of X-over P1 (loxP) sequence, a flippase recognition target (FRT) sequence, a rox sequence, an attachment-phage (attP) sequence, an attachment-bacteria (attB) sequence, an attachment-left (attL) sequence, an attachment-right (attR) sequence, a VloxP sequence, a vox sequence or a transposase recognition inverted terminal repeat (ITR).

Embodiment 41: The method of any one of Embodiments 31-40, wherein the cell is a bacterial cell, a plant cell, or a mammalian cell.

Embodiment 42: The method of any one of Embodiments 31-41, wherein the cell is an isolated cell, a cell from a cell line, a primary cell, or a cell in a subject.

Embodiment 43: The method of any one of Embodiments 31-42, wherein the cell is a pluripotent stem cell, an induced pluripotent stem cell (iPSCs), a hematopoietic stem cell (HSCs), a B-cell, a T-cell, a natural killer (NK) cell, or a terminally differentiated non-dividing cell, such as neuron, muscle, or hepatocyte.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A circular single-stranded DNA (cssDNA) molecule comprising a recombinase recognition sequence recognized by a recombinase.

2. The cssDNA molecule of claim 1, wherein at least one of the following applies:
(a) the recombinase is a site-specific recombinase (SSR),
(b) the site-specific recombinase is a tyrosine integrase, a serine integrase, or a site-specific transposase,
(c) the recombinase recognition sequence is a locus of X-over P1 (loxP) sequence, a flippase recognition target (FRT) sequence, a rox sequence, an attachment-phage (attP) sequence, an attachment-bacteria (attB) sequence, an attachment-left (attL) sequence, an attachment-right (attR) sequence, a VloxP sequence, a vox sequence, or a transposase recognition inverted terminal repeat (ITR),
(d) the cssDNA molecule further comprises a payload sequence, optionally wherein the payload sequence encodes an RNA molecule or a polypeptide, optionally wherein the payload sequence comprises a length of about 1 nucleotide to about 50,000 nucleotides,
(e) the cssDNA molecule is purified or enriched.

3. (canceled)

4. (canceled)

5. (canceled)

6. (canceled)

7. (canceled)

8. (canceled)

9. A double stranded DNA (dsDNA) molecule comprising:
a template sequence encoding a cssDNA comprising a recombinase recognition sequence recognized by a recombinase; and
a cssDNA conversion sequence.

10. The dsDNA molecule of claim 9, wherein at least one of the following applies:
(a) the double stranded DNA molecule is a linear dsDNA molecule or a circular dsDNA molecule,
(b) the cssDNA conversion sequence is a sequence originated or derived from an M13 phage f1 origin, an M13 phage replication initiator, an M13 phage replication terminator, or an M13 phage packing signal (PS),
(c) the dsDNA molecule further comprises a payload sequence, or a payload sequence insertion site for inserting the payload sequence, optionally wherein the payload insertion site is a multiple cloning site,
(d) the dsDNA molecule is one single DNA molecule, or two or more separate DNA molecules.

11. (canceled)

12. (canceled)

13. (canceled)

14. (canceled)

15. A kit comprising:
the cssDNA molecule of claim 1; and
a recombinase or a nucleic acid encoding the recombinase,
wherein the recombinase recognizes the recombinase recognition sequence of the cssDNA.

16. The kit of claim 15, wherein at least one of the following applies:
(a) the recombinase is a serine integrase, a tyrosine integrase, or a site-specific transposase,
(b) the recombinase comprises at least one selected from the group consisting of Cre, Flp, Dre, SCre, VCre, Vika, B2, B3, KD, ΦC31, Bxb1, RDF, λ, HK022, HP1, γδ, ParA, Gin, R4, TP901-1, TG1, PhiRv1, PhiBT1, SprA, XisF, TnpX, R, A118, spoIVCA, MR11, SCCmec, TndX, XerC, XerD, XisA, Hin, Cin, mrpA, beta, PhiFC1, Fre, Clp, sTre, FimE, HbiF, φFC1, φC1, q 370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), Minos, R1, R2, R3, R4, R5, Mu transposase, Tn3 transposase, Tn5 transposase, Tn7 transposase, Tol2, Sleeping Beauty transposase, and PiggyBac transposase,
(c) the kit comprises the nucleic acid encoding the recombinase, wherein the nucleic acid encoding the recombinase is an mRNA, a single stranded DNA, or a double stranded DNA,
(d) the kit comprises the nucleic acid encoding the recombinase, and wherein the nucleic acid encoding the recombinase is part of the cssDNA molecule or the dsDNA molecule,
(e) the kit further comprises a component for introducing the cssDNA, and the recombinase or the nucleic acid encoding the recombinase, into the cell.

17. (canceled)

18. (canceled)

19. The kit of claim 15, further comprising an oligonucleotide fully or partially complementary with the cssDNA molecule.

20. The kit of claim 19, wherein the oligonucleotide is a primer that converts the cssDNA molecule to a circular double stranded DNA molecule inside a cell, optionally wherein the oligonucleotide comprises a length of about 5 nucleotides to about 200 nucleotides.

21. (canceled)

22. (canceled)

23. (canceled)

24. A system comprising:
the kit of claim 15; and
a cell to be engineered.

25. The system of claim 24, wherein at least one of the following applies:
(a) a genomic DNA or a mitochondrial DNA of the cell comprises a landing pad sequence for the integration of the cssDNA molecule into the genomic DNA or the mitochondrial DNA,
(b) the cell is a bacterial cell, a plant cell, or a mammalian cell,
(c) the cell is an isolated cell, a cell from a cell line, a primary cell, or a cell in a subject,
(d) the cell is a pluripotent stem cell, an induced pluripotent stem cell (iPSCs), a hematopoietic stem cell (HSCs), a B-cell, a T-cell, a natural killer (NK) cell, or a terminally differentiated non-dividing cell, optionally wherein the terminally differentiated non-dividing cell is a neuron, a muscle, or a hepatocyte.

26. The system of claim 25, wherein the landing pad sequence is recognized by the recombinase such that the recombinase opens up the cssDNA molecule and inserts the linearized cssDNA molecule into the genomic DNA or the mitochondrial DNA using the landing pad sequence as an anchor site, optionally the landing pad sequence is a locus of X-over P1 (loxP) sequence, a flippase recognition target (FRT) sequence, a rox sequence, an attachment-phage (attP) sequence, an attachment-bacteria (attB) sequence, an attachment-left (attL) sequence, an attachment-right (attR) sequence, a VloxP sequence, a vox sequence, or a transposase recognition inverted terminal repeat (ITR).

27. (canceled)

28. (canceled)

29. (canceled)

30. (canceled)

31. A method for performing genetic engineering in a cell, comprising introducing into the cell:
the cssDNA of claim 1; and
a recombinase or a nucleic acid encoding the recombinase,
wherein the recombinase recognizes the recombinase recognition sequence of the cssDNA to integrate the cssDNA into a genomic DNA or a mitochondrial DNA of the cell.

32. The method of claim 31, wherein at least one of the following applies:
(a) the recombinase is a serine recombinase, a tyrosine recombinase, or a site-specific transposase,
(b) the recombinase is Cre, Flp, Dre, SCre, VCre, Vika, B2, B3, KD, ΦC31, Bxb1, RDF, λ, HK022, HP1, γδ, ParA, Gin, R4, TP901-1, TG1, PhiRv1, PhiBT1, SprA, XisF, TnpX, R, A118, spoIVCA, MR11, SCCmec, TndX, XerC, XerD, XisA, Hin, Cin, mrpA, beta, PhiFC1, Fre, Clp, sTre, FimE, HbiF, φFC1, φC1, q 370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), Minos, R1, R2, R3, R4, R5, Mu transposase, Tn3 transposase, Tn5 transposase, Tn7 transposase, Tol2, Sleeping Beauty transposase, or PiggyBac transposase,
(c) the method comprises introducing into cells a sense stranded cssDNA, an antisense stranded cssDNA, or a mixture of both,
(d) the cell is a bacterial cell, a plant cell, or a mammalian cell,
(e) the cell is an isolated cell, a cell from a cell line, a primary cell, or a cell in a subject,
(f) the cell is a pluripotent stem cell, an induced pluripotent stem cell (iPSCs), a hematopoietic stem cell (HSCs), a B-cell, a T-cell, a natural killer (NK) cell, or a terminally differentiated non-dividing cell, optionally wherein the terminally differentiated non-dividing cell is a neuron, muscle, or hepatocyte.

33. (canceled)

34. The method of claim 31, further comprising introducing into the cell an oligonucleotide fully or partially complementary to at least a portion of the cssDNA molecule.

35. The method of claim 34, wherein a cellular machinery of the cell converts the cssDNA molecule into a circular double stranded DNA molecule using the oligonucleotide as a primer.

36. The method of claim 34, wherein the oligonucleotide comprises a length of about 5 nucleotides to about 200 nucleotides.

37. (canceled)

38. The method of claim 31, wherein the genomic DNA or the mitochondrial DNA of the cell comprises a landing pad sequence for the integration of the cssDNA molecule into the genomic DNA or the mitochondrial DNA.

39. The method of claim 38, wherein at least one of the following applies:
(a) further comprising culturing the cell under a condition sufficient for the landing pad sequence to be recognized by the recombinase and for the recombinase to open up the cssDNA molecule and insert the linearized cssDNA molecule into the genomic DNA or the mitochondrial DNA using the landing pad sequence as an anchor site,
(b) the landing pad sequence is a locus of X-over P1 (loxP) sequence, a flippase recognition target (FRT) sequence, a rox sequence, an attachment-phage (attP) sequence, an attachment-bacteria (attB) sequence, an attachment-left (attL) sequence, an attachment-right (attR) sequence, a VloxP sequence, a vox sequence, or a transposase recognition inverted terminal repeat (ITR).

40. (canceled)

41. (canceled)

42. (canceled)

43. (canceled)


SEQUENCE LISTING (9 sequences)

SEQ ID NO: 1 - DNA, 52 bp, synthetic construct
  source 1..52: mol_type = other DNA
gtggtttgtctggtcaaccaccgcggtctcagtggtgtacggtacaaaccca

SEQ ID NO: 2 - DNA, 46 bp, synthetic construct
  source 1..46: mol_type = other DNA
ggccggcttgtcgacgacggcggtctccgtcgtcaggatcatccgg

SEQ ID NO: 3 - DNA, 52 bp, synthetic construct
  source 1..52: mol_type = other DNA
tgggtttgtaccgtacaccactgagaccgcggtggttgaccagacaaaccac

SEQ ID NO: 4 - DNA, 46 bp, synthetic construct
  source 1..46: mol_type = other DNA
ccggatgatcctgacgacggagaccgccgtcgtcgacaagccggcc

SEQ ID NO: 5 - RNA, 20 nt, synthetic construct
  source 1..20: mol_type = other RNA
ggtagtcgtactcgtcgtcg

SEQ ID NO: 6 - DNA, 20 bp, synthetic construct
  source 1..20: mol_type = other DNA
cagcccttcctttttgtgcg

SEQ ID NO: 7 - DNA, 20 bp, synthetic construct
  source 1..20: mol_type = other DNA
ggggaaactgagtcctggaa

SEQ ID NO: 8 - DNA, 2334 bp, synthetic construct
  source 1..2334: mol_type = other DNA
ttaaccctagaaagatagtctgcgtaaaattgacgcatgcattcttggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcttggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccatactgagtgggtggagactgaagttaggccagcttggcacttgatgtaattctccttggaatttgccctttttgagtttggatcttggttcattctcaagcctcagacagtggttcaaagtttttttcttccatttaaggtgtcgtgaacgcgtattaatacgctgctagcggtaccgccaccatgggatcgggtgggactagtggcagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcgccacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggccaacttcaagatccgccacaacgtggaggacggcagcgtgcagctcgccgaccactaccagcagaacacccccatcggcgacggccccgtgctgctgcccgacaaccactacctgagcacccagtccgtgctgagcaaagaccccaacgagaagcgcgatcacatggtcctgctggagttcgtgaccgccgccgggatcactggaaccggtgctggaagtggttgataagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcagaccgataaaacacatgcgtcaattttacgcatgattatctttaacgtacgtcacaatatgattatctttctagggttaa

SEQ ID NO: 9 - DNA, 2080 bp, synthetic construct
  source 1..2080: mol_type = other DNA
atggccccaaaaaagaaaagaaaagtgtatccctatgatgtccccgattatgccggttcaggctctagcctggacgacgagcacatcctgagcgccctgctgcagagcgacgacgaactggtgggcgaggacagcgacagcgaggtcagcgaccacgtgtccgaggacgacgtgcagtccgacaccgaggaagccttcatcgacgaggtgcacgaagtgcagcctaccagcagcggctccgagatcctggacgagcagaacgtgatcgagcagcctggcagctccctggccagcaacagaatcctgaccctgccccagagaaccatcagaggcaagaacaagcactgctggtccacctccaagagcaccaggcggagcagagtgtccgccctgaacatcgtgcggagccagaggggccccaccagaatgtgcagaaacatctacgaccccctgctgtgcttcaagctgttcttcaccgacgagatcatcagcgagatcgtgaagtggaccaacgccgagatcagcctgaagaggcgggagagcatgaccagcgccaccttcagagacaccaacgaggacgagatctacgccttcttcggcatcctggtgatgaccgccgtgagaaaggacaaccacatgagcaccgacgacctgttcgacagatccctgagcatggtgtacgtgtccgtgatgagcagagacagattcgacttcctgatcagatgcctgagaatggacgacaagagcatcagacccaccctgcgggagaacgacgtgttcacccccgtgcggaagatctgggacctgttcatccaccagtgcatccagaactacacccctggcgcccacctgaccatcgatgagcagctgctgggcttcagaggcagatgccccttcagagtgtacatccccaacaagcccagcaagtacggcatcaagatcctgatgatgtgcgacagcggcaccaagtacatgatcaacggcatgccctacctgggcagaggcacccagacaaacggcgtgcccctgggcgagtactacgtgaaagaactgagcaagcctgtgcatggcagctgcaggaacatcacctgcgacaactggttcaccagcatccccctggccaagaacctgctgcaggaaccctacaagctgaccatcgtgggcaccgtgcggagcaacaagcgggagatcccagaggtgctgaagaacagcagatccagacctgtgggaacaagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacagatggcccatggccctgctgtacggcatgatcaatatcgcctgcatcaacagcttcatcatctacagccacaacgtgtccagcaagggcgagaaggtgcagagccggaagaaattcatgcggaacctgtacatgagcctgacctccagcttcatgagaaagagactggaagcccccaccctgaagagatacctgcgggacaacatcagcaacatcctgcccaaggaagtgccaggaacaagcgacgacagcaccgaggaacccgtgatgaagaagaggacctactgcacctactgtcccagcaagatcagaagaaaggccaacgccagctgcaagaaatgcaaaaaagtgatctgccgggagcacaacatcgacatgtgccagagctgtttctgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga